(19) United States
(12) Patent Application Publication
(10) Pub. No.: US 2024/0335324 A1
(43) Pub. Date: Oct. 10, 2024

(54) SINGLE AND/OR DUAL (532 AND/OR 577) nm LASER DELIVERY SYSTEM ATTACHED TO AN OPHTHALMIC MICROSCOPE

(71) Applicant: APPASAMY ASSOCIATES PVT LTD, Chennai (IN)

(72) Inventors: Narayanasamy RAVILLA KASTHURI, Chennai (IN); Sri Rama Prasad JEYARAJ, Chennai (IN)

(21) Appl. No.: 18/294,321

(22) PCT Filed: Oct. 13, 2022
(86) PCT No.: PCT/IN2022/050912

§ 371 Date: Feb. 1, 2024

(30) Foreign Application Priority Data
Oct. 14, 2021 (IN) 202141031550

(51) Int. Cl. A61F 9/008; A61B 18/00; A61B 18/20; A61B 90/20

(52) U.S. Cl. CPC A61F 9/0084; A61B 90/20; A61F 9/00834; A61B 2018/00601; A61B 2018/2025; A61B 2018/20355; A61B 2018/20553; A61B 2018/207; A61F 2009/0087

(57) ABSTRACT

The present invention relates to a single and/or dual laser delivery system attached to an existing ophthalmic operating microscope, comprising of a single and/or dual wavelength laser unit [1]; and a delivery system [5]. The laser unit [1] comprises of a green laser module 532 nm [3] and a yellow laser module 577 nm [4] along with an aiming beam [7] 635 nm. The delivery system comprises of a right-angle prism [5A]: X and Y Galvo assembly [5B] and a focusing lens assembly [5C]. The laser unit passes through a fiber optic cable [6] gets reflected using the right angle prism, gets deflected by the Galvo assembly, enters the focusing lens assembly and gets reflected by a folding mirror [5D] and focused on human eye capsule. The system converges and focuses the laser wavelengths in line with the aiming beam to create a circular pattern to accomplish a pre-measured Capsulorhexis.

### FIELD OF INVENTION

The present invention relates to a laser unit, particularly to single and/or dual (532 and/or 577) nm laser delivery system attached to an existing ophthalmic operating microscope for creation of a Capsulorhexis in a human eye.

### BACKGROUND OF INVENTION

Capsulorhexis generally refer to removal of a part of the capsule by shear and stretch forces, but in situations like a developmental cataract a part of the capsule is also removed by a similar technique. A cataract is a dense, cloudy area that forms in the lens of an eye. Creation of the Capsulorhexis is a key stage in cataract surgery, and producing a precise rhexis is of paramount importance. Capsulorhexis was first performed by removing the lens and safely implanting intraocular lens (IOL) in the capsular bag. This procedure avoided the radial tears which occurred frequently in the can-opener capsulotomies that were performed at the time. A correctly sized, and a well-centered Capsulorhexis with a 360° overlapping capsular edge prevents IOL tilt, myopic shift, optic de-centration, and posterior and anterior capsular opacification, partly due to a combination of the symmetric contractile forces of the capsular bag, and the shrink wrap effect. However, eccentric or irregularly shaped Capsulorhexis with a diameter extending beyond the optic edge may lose these advantages.

Currently, Capsulorherxis step is being done during Phacosurgery either by manually using a 26G needle or forceps. The selection depends on the length of the incision that the surgeon chooses to make. However, the manual procedure is time consuming and also involves surgeon's skill. Also, in manual procedure, it is essential that capsule tear direction is controlled, as the overall direction of pulling forces on the capsule flap can cause different tearing direction patterns. Recently Capsulorherxis step is being done using a Femtosecond laser which is a newer technology and very expensive investment too for individual practitioners. Further, it also requires a separate specialized operation room with accurately maintained room temperature for its perfect functioning hence very high recurring maintenance cost. However, in the Femtosecond laser assisted cataract surgery, the laser equipment and the ultrasonic emulsification equipment are not integrated, the operation process needs to be carried out separately in two different operation rooms, which also involves shifting of the patient from one operating room to an another operating room and hence the operation time is prolonged. In addition, patients with primary diseases of small pupils and corneal epithelium and adverse eye socket shapes to the connection of laser equipment and eyes are not suitable for Femtosecond laser-assisted cataract surgery.

U.S. Ser. No. 11/083,625B2 discloses a system for laser ophthalmic surgery comprising of laser assembly, confocal detection assembly and scanning assembly. The system has used two treatment lasers.

First treatment laser is infrared wavelength 870 nm to 1200 nm and second treatment laser is ultraviolet wavelength 320 nm to 370 nm. The focal spot size is 150 microns.

U.S. Pat. No. 9,895,056B2 discloses an ophthalmic laser illuminator with three different wavelength lasers Red (At least 620 nm), Blue (less than 480 nm), Green (between 552 nm and 557 nm). The illuminator has mixer/homogenizer for mixing the three wavelengths of light.

U.S. Pat. No. 8,315,280B2 relates to multiple wavelength laser system which includes at least two semiconductor diode lasers and one light guide system. The wavelength ranges are 480 nm to 520 nm, 530 to 550 nm, 560 to 580 nm and 1040 nm to 1070 nm. However, the purpose is for therapeutic sectors that include photocoagulation, transpupillary thermotherapy, photothermal tumor treatment and photodynamic therapy.

U.S. Pat. No. 5,426,662A discloses a laser system for ophthalmological treatments with two competing wavelength regions. The wavelength regions are reflective at 647 nm and transmissive at 531 nm and 568 nm.

U.S. Ser. No. 10/022,270B2 discloses methods and systems for performing laser-assisted surgery. The wavelength range is from 500 nm to 1100 nm and the focal spot has a diameter of 1 to 20 microns.

U.S. Ser. No. 10/596,037B2 relates to an ophthalmic phototherapy device which promotes the healing of damaged or diseased eye tissue. The system uses multiple wavelengths and the ranges are 500 nm to 650 nm or 640 nm to 700 nm or 800 nm to 900 nm.

WO2015131135A1 discloses laser assisted cataract surgery methods and devices which utilize one or more treatment laser beams to create a shaped opening in the anterior lens capsule of the eye when performing a capsulorrhexis procedure. The laser may operate at a wavelength of about 577 nm or about 590 nm or about 810 nm. The spot size of laser is 100 microns to 350 microns.

KR102190881B1 discloses a laser device for high-intensity pain treatment using a composite wavelength and a programmed scan handpiece. The wavelength band of the laser beam is any one of 980 nm and 1064 nm in the case of a short wavelength, and provides a combined wavelength of 980 nm and 1064 nm in the case of a complex wavelength.

WO1993001638A1 discloses a laser system with dielectric mirror, laser medium and motor driven carriage. The laser medium utilizing a YAG type crystal to make it possible, the generation of wavelengths of 1064 nm, 1320 nm, 532 nm and 660 nm from a single laser system.

KR101177478B1 discloses a laser apparatus with laser oscillating unit, power converting unit, an optical coupling unit, laser output cut-off unit and a scope unit to controls the direction or spot size of a laser beam. The laser apparatus use dual wavelength namely yellow wavelength (578 nm) and green wavelength (511 nm).

Accordingly, there exists a need for a compact, precision, safety and cost effective single and/or dual wavelength laser unit with a delivery system that could be retrofitted to an existing ophthalmic operating microscope to overcome the afore-mentioned drawbacks.

### OBJECTS OF INVENTION

One or more of the problems of the conventional prior art may be overcome by various embodiments of the system and method of the present invention.

It is the primary object of the present invention to provide a compact, cost effective single and/or dual wavelength laser unit with a delivery system attached to an existing ophthalmic operating microscope.

It is another object of the present invention to provide a single and/or dual wavelength laser delivery system attached to an existing ophthalmic operating microscope to create a circular pattern to accomplish a pre-measured diameter size of Capsulorhexis with a precise circular cut of the capsule in human eye with a precise diameter of 3 mm to 6 mm in steps of 0.5 mm.

It is another object of the present invention to provide a Galvo assembly driven single and/or dual wavelength laser delivery system attached to an existing ophthalmic operating microscope for laser beam to be focused on to the capsular bag in the human eye.

It is another object of the present invention, wherein the laser delivery system converges and focuses the single and/or dual wavelength laser beam to create a circular pattern in order to accomplish a pre-measured Capsulorhexis with a precise circular cut of the capsule in the human eye with a precise diameter of 3 mm to 6 mm in steps of 0.5 mm.

It is another object of the present invention, wherein the laser delivery system has an aiming beam which is used to locate and guide user to aim the single and/or dual wavelength laser beam before the Capsulorhexis.

### SUMMARY OF INVENTION

Thus according to the basic aspect of the present invention, there is provided a single and/or dual wavelength laser delivery system attached to an existing ophthalmic operating microscope, said system comprises of:
a single and/or dual wavelength laser unit;
a delivery system; and
an ophthalmic operating microscope,
wherein the single and/or dual wavelength laser unit comprises of a green laser module 532 nm and a yellow laser module 577 nm along with an aiming beam 635 nm within both the laser modules to create a circular pattern with a precise circular cut of the capsule in a human eye,
wherein the aiming beam 635 nm is used to locate and guide user to aim the 535 nm and/or 577 nm laser beam,
wherein each of the laser modules comprises of a low pass filter and a focusing lens respectively,
wherein the green and yellow laser modules are connected to the delivery system through an interchangeable single fiber optic cable,
wherein the delivery system comprises of a right-angle prism; a X and Y Galvo assembly and a focusing lens assembly,
wherein the single and/or dual wavelength module from the laser unit passes through the fiber optic cable gets reflected using the right angle prism, gets deflected by the X and Y Galvo assembly, enters the focusing lens assembly and gets reflected by a folding mirror and focused on the human eye capsule at 90 degrees, and
wherein said laser delivery system attached to the operating microscope's converges and focuses the laser wavelength of 532 nm and/or 577 nm laser beam in-line with the 635 nm aiming beam to create a circular pattern to accomplish a pre-measured Capsulorhexis with a precise circular cut of the capsule in the human eye.

It is another aspect of the present invention, wherein the low pass filters are mounted at an angle that transmits the 532 nm green laser or 577 nm yellow laser beam and reflects the 635 nm aiming beam.

It is another aspect of the present invention, wherein the laser beam from the fiber optic cable passes through the focusing lens assembly to get a required spot size of 100 microns to 300 microns.

It is another aspect of the present invention, wherein the folding mirror is mounted in an angle of 45 degree with a tolerance level of + or −2 degrees along the laser optical axis.

It is another aspect of the present invention, wherein the single and/or dual wavelength laser delivery system converges and focuses the laser wavelength of 532 nm and/or 577 nm laser beam in-line with the 635 nm aiming beam to create a circular pattern to accomplish a pre-measured Capsulorhexis with a precise circular cut of the capsule in the human eye with a precise diameter of 3 mm to 6 mm in steps of 0.5 mm.

It is another aspect of the present invention, wherein the laser unit comprises a foot switch to trigger the 532 nm and/or 577 nm laser beam.

### BRIEF DESCRIPTION OF THE DRAWINGS

FIG. 1: illustrates single and/or dual wavelength laser delivery system according to the present invention.

FIG. 2: illustrates block diagram of single and/or dual wavelength laser delivery system according to the present invention.

FIG. 3: illustrates laser unit of the system according to the present invention.

FIG. 4: illustrates single and/or dual wavelength laser delivery system attached to an existing ophthalmic operating microscope according to the present invention.

FIG. 5: illustrates delivery system and laser beams according to the present invention.

FIG. 6: illustrates the aiming beam RED laser spot and circular diameter of 5.5 mm.

FIG. 7: illustrates the proof of conformity in lab trials of Capsulorhexis in animal eye samples for 5.5 mm and 6 mm diameter.

FIG. 8: illustrates the proof of Capsulorhexis trials conducted on animal eyes.

### DETAILED DESCRIPTION OF THE INVENTION WITH REFERENCE TO THE ACCOMPANYING FIGURES

The present invention as herein described relates to a single and/or dual (532 and/or 577) nm laser delivery system attached to an existing ophthalmic operating microscope for creation of circular pattern to accomplish a pre-measured diameter size of capsulorhexis.

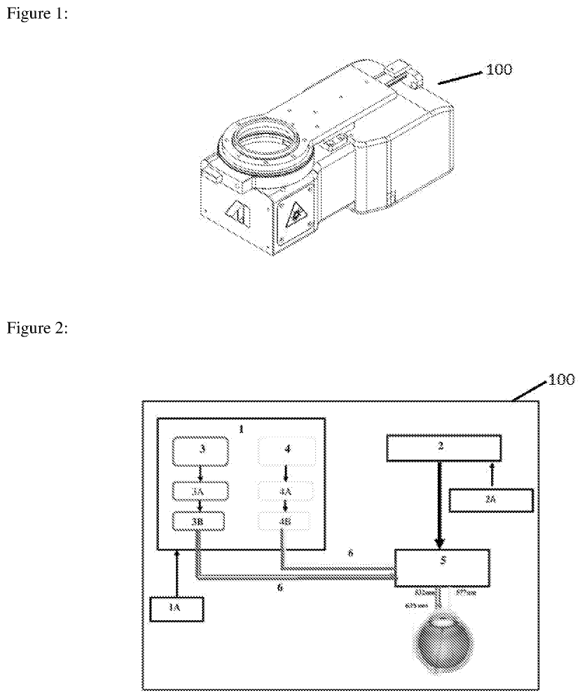

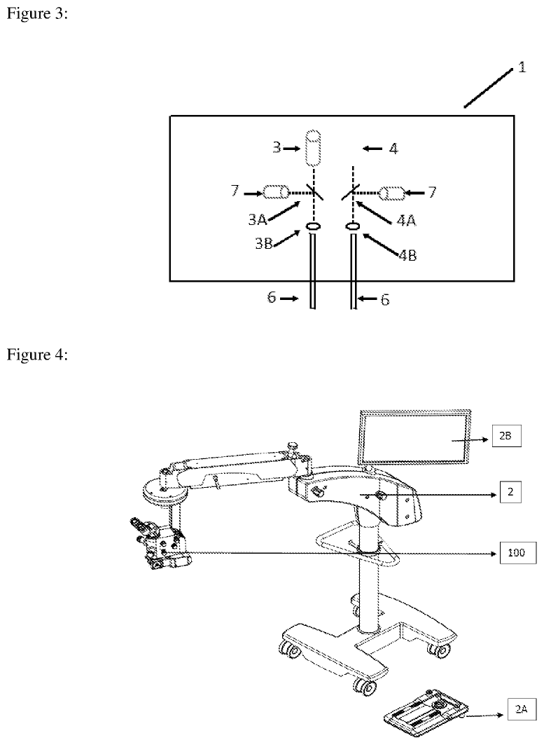

Referring to FIGS. 1 to 3, the single and/or dual wavelength laser delivery system [100] comprises of a single and/or dual wavelength laser unit [1]; a delivery system [5]; and an ophthalmic operating microscope [2]. The laser unit [1] comprises of a green laser module 532 nm [3] and a yellow laser module 577 nm [4] along with 635 nm aiming beam [7] (as shown in FIG. 3) within both the laser modules [3 and 4] to create a circular pattern with a precise circular cut of the capsule in the human eye. The 635 nm aiming beam [7] is used to locate and guide the user to aim the 535 nm and/or 577 nm laser beam on the human eye capsule before the Capsulorhexis. The green laser module [3] comprises of a low pass filter [3A] and a focusing lens [3B]. The yellow laser module [4] comprises of a low pass filter [4A] and a focusing lens [4B]. The low pass filters [3A and 4A] are mounted at an angle that transmits the 532 nm green laser [3] or 577 nm yellow laser beam [4] and reflects the 635 nm aiming beam [7]. The laser modules [3 and 4] are connected to the delivery system [5] through an interchangeable single fiber optic cable [6]. The laser unit [1] includes a foot switch [1A] to trigger the laser beam.

Referring to FIG. 4, the single and/or dual wavelength laser delivery system [100] is positioned and fixed to the operating microscope's [2] optical head. Said laser delivery system [100] converges and focuses laser wavelength of 532 nm and/or 577 nm laser beam in-line with 635 nm aiming beam to create a circular pattern in order to accomplish a pre-measured Capsulorhexis with a precise circular cut of the capsule in the human eye. The ophthalmic microscope [2] includes a foot switch [2A] for focus adjustment and magnification changes of the operating microscope. The ophthalmic microscope [2] includes a display unit [2B] to display the live image of Capsulorhexis procedure.

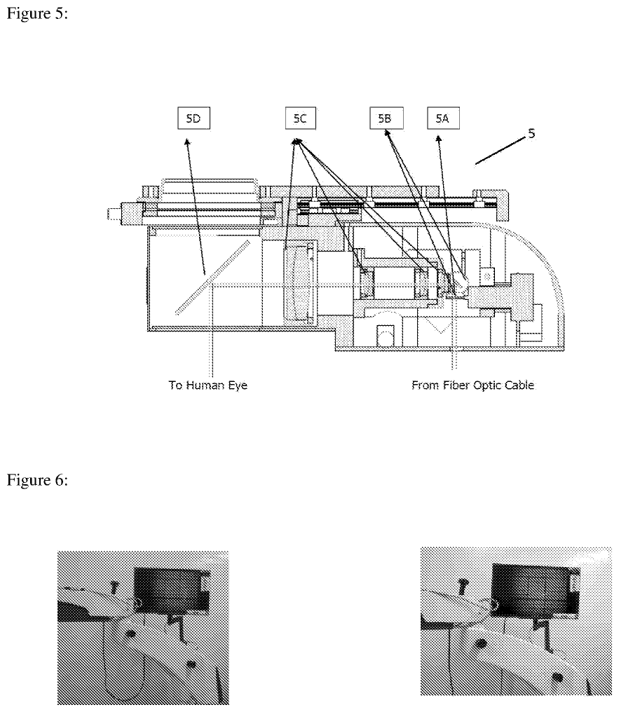

Referring to FIG. 5, the delivery system [5] comprises of a right-angle prism [5A] and an X and Y Galvo assembly [5B] to generate required laser deflection and a circular laser pattern. The laser beam from the fiber optic cable [6] passes through a focusing lens assembly [5C] to get a required spot size of 100 microns to 300 microns. The laser beam is focused on the human eye using a folding mirror [5D]. The focusing lens assembly [5C] includes four focusing lenses. The folding mirror [5D] is at along the laser optical axis that deflects the laser beam 90 degrees into the human eye capsule to perform the Capsulorhexis. In an aspect, the folding mirror [5D] is mounted on an optics assembly in an angle of 45 degree with a tolerance level of + or −2 degrees.

The delivery system [5] converges and focuses laser wavelength of 532 nm and/or 577 nm laser beam to create a circular pattern in order to accomplish a pre-measured Capsulorhexis with a precise circular cut of the capsule in the human eye from diameter 3 mm to 6 mm in steps of 0.5 mm. FIG. 6 shows the aiming beam RED laser spot and circular diameter of 5.5 mm.

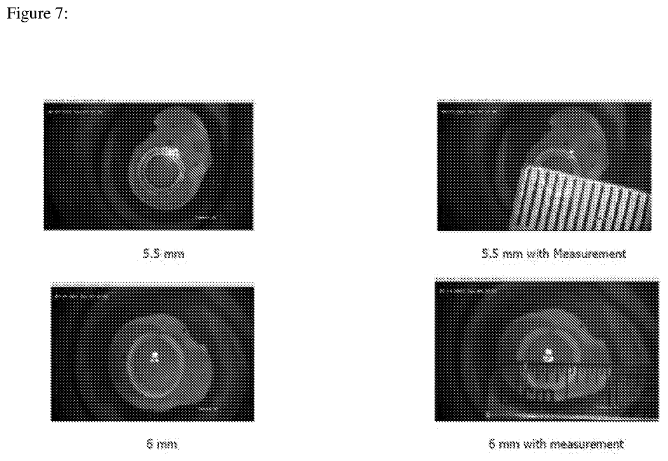

A number of trials of capsulorhexis in animal eyes have been conducted using the single and/or dual (532 and/or 577) nm laser delivery system and have achieved the capsulorhexis in the diameter of 5.5 mm and 6 mm. FIG. 7 illustrates the proof of conformity in lab trials of Capsulorhexis in animal eye samples for 5.5 mm and 6 mm diameter.

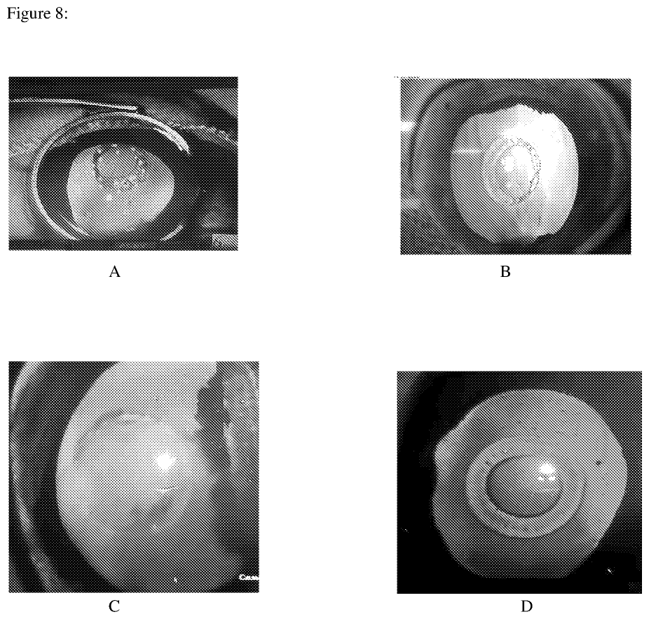

Animal eye lab trials were being conducted from 2020, and have completed 17 trials so far. This process was evolved using different laser powers, laser ON time duration. Standard ophthalmic viscoelastic solution was used to protect the Corneal endothelium cells during the laser ablation procedure and for better capsule identification ophthalmic blue dye solution is used for staining the capsule. Also different concentration of the Ophthalmic blue dye solution was used to observe the absorption level of the two different laser wavelengths (532/577) nm on the capsule tissue under study. FIG. 8A shows the first trial; 8B shows the 10^{th} trial; 8C shows the 12^{th} trial and 8D shows the 15^{th} trial.

The single and/or dual wavelength laser delivery system [100] could be retrofitted to an existing ophthalmic microscope; said system is mounted along the optical axis to suit the requirement, so that it does not obscure the Surgeon's view while operating. The single and/or dual wavelength laser delivery system [100] which could be retrofitted to an existing ophthalmic microscope saves Surgeon time since Patient is not required to be shifted from one Surgical equipment to the other Surgical equipment after the Capsulorhexis is completed, produce accuracy and repeatability in creation of a precise diameter of the capsulorhexis from diameter 3 mm to 6 mm in steps of 0.5 mm. Advantageously, the single and/or dual wavelength laser delivery system [100] along with the aiming beam provides flexibility and compatibility to the ophthalmic Surgeons.

### Working:

For cataract surgery in a human eye, the crystalline lens is replaced with an Intraocular Lens (IOL). The crystalline lens is enclosed in a capsule on front and back side. In conventional cataract surgery, the front portion (anterior) of capsule is circularly removed by 26g needle or capsular forceps and IOL is placed. The present invention is to a system [100] which performs laser assisted procedure to speed up the Cataract surgery of removing the anterior of capsule from diameter 3 mm to 6 mm. In an aspect, anyone of the two wavelengths 532 nm [3] or 577 nm [4] at a given point of time selected by the user is used along with guiding/aiming beam [7] of wavelength 635 nm. The 532 nm or 577 nm is generated in the laser unit [1]. The 532 nm or 577 nm laser along with aiming beam is coupled by focusing lens while leaving the laser unit. The 532 nm or 577 nm laser will enter one end of the fiber optic cable [6] which works in total internal reflection principle without loss and leave the other end. The other end of the fiber optic cable [6] is connected to the delivery system [5] which is mounted in the operating microscope [2]. These lasers leaving the other end of the fiber optic cable [6] passes through the right angle prism [5A], gets deflected by the X and Y Galvo assembly [5B], enters the focusing lens assembly [5C]. These lasers after passing through the focusing lens assembly [5C] is reflected/folded by the folding mirror [5D] and focused on the human eye capsule. By choosing the required laser 532 nm and/or 577 nm and diameter and proper focusing of the microscope, the Capsulorhexis is performed using the foot switch [1A].

We claim:

1. A single and/or dual wavelength laser delivery system [100] attached to an existing ophthalmic operating microscope, said system comprises of:
a single and/or dual wavelength laser unit [1];
a delivery system [5]; and
an ophthalmic operating microscope [2],
wherein the single and/or dual wavelength laser unit [1] comprises of a green laser module 532 nm [3] and a yellow laser module 577 nm [4] along with an aiming beam [7] 635 nm within both the laser modules [3 and 4] to create a circular pattern with a precise circular cut of the capsule in a human eye,
wherein the aiming beam 635 nm [7] is used to locate and guide user to aim the 535 nm and/or 577 nm laser beam,
wherein each of the laser modules [3 and 4] comprises of a low pass filter [3A and 4A] and a focusing lens [3B and 4B] respectively,
wherein the green and yellow laser modules [3 and 4] are connected to the delivery system [5] through an interchangeable single fiber optic cable [6],
wherein the delivery system [5] comprises of a right-angle prism [5A]; a X and Y Galvo assembly [5B] and a focusing lens assembly [5C],
wherein the single and/or dual wavelength module from the laser unit [1] passes through the fiber optic cable [6] gets reflected using the right angle prism [5A], gets deflected by the X and Y Galvo assembly [5B], enters the focusing lens assembly [5C] and gets reflected by a folding mirror [5D] and focused on the human eye capsule at 90 degrees, and
wherein said laser delivery system [100] attached to the operating microscope's [2] converges and focuses the laser wavelength of 532 nm and/or 577 nm laser beam in-line with the 635 nm aiming beam [7] to create a circular pattern to accomplish a pre-measured Capsulorhexis with a precise circular cut of the capsule in the human eye.

2. The single and/or dual wavelength laser delivery system [100] as claimed in claim 1, wherein the low pass filters [3A and 4A] are mounted at an angle that transmits the 532 nm green laser [3] or 577 nm yellow laser beam [4] and reflects the 635 nm aiming beam [7].

3. The single and/or dual wavelength laser delivery system [100] as claimed in claim 1, wherein the laser beam from the fiber optic cable [6] passes through the focusing lens assembly [5C] to get a required spot size of 100 microns to 300 microns.

4. The single and/or dual wavelength laser delivery system [100] as claimed in claim 1, wherein the folding mirror [5D] is mounted in an angle of 45 degree with a tolerance level of + or −2 degrees along the laser optical axis.

5. The single and/or dual wavelength laser delivery system [100] as claimed in claim 1 converges and focuses the laser wavelength of 532 nm and/or 577 nm laser beam in-line with the 635 nm aiming beam [7] to create a circular pattern to accomplish a pre-measured Capsulorhexis with a precise circular cut of the capsule in the human eye with a precise diameter of 3 mm to 6 mm in steps of 0.5 mm.

6. The single and/or dual wavelength laser delivery system [100] as claimed in claim 1, wherein the laser unit [1] comprises a foot switch [1A] to trigger the 532 nm and/or 577 nm laser beam.
